(19) United States
(12) Patent Application Publication
(10) Pub. No.: US 2009/0304591 A1
(43) Pub. Date: Dec. 10, 2009

(54) METHOD AND DEVICE FOR OPTICAL DETECTION OF THE EYE

(76) Inventors: Christoph Russmann, Jena (DE); Manfred Dick, Gefell (DE)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 12/306,882

(22) PCT Filed: Jun. 23, 2007
(86) PCT No.: PCT/EP07/05555

§ 371 Date: Dec. 29, 2008

(30) Foreign Application Priority Data
Jun. 29, 2006 (DE) 10 2006 030 382.2

(51) Int. Cl. A61K 49/00; A61P 27/02; A61B 6/00

(52) U.S. Cl. 424/9.1; 600/476

(57) ABSTRACT

A solution for optical detection of the eye. Molecular markers are used for high-contrast diagnosis of eye diseases, other diseases, and other vital parameters which can be diagnosed in the eye. For optical detection of the eye, a molecular marker with spectral characteristics of absorption and/or scattering in the visual and infrared spectral region is introduced into the eye and bound to a specific target. The interaction of the molecular marker with the target is detected by means of optical imaging methods, such as fundus photography, confocal laser microscopy, polarisation-optical imaging methods, holographic methods or especially OCT methods. The use of optical methods is strongly preferred for the diagnosis of the eye as a result of the high transparency of the optical system of the eye compared to other body parts.

The present application is a National Phase entry of PCT Application No. PCT/EP2007/005555, filed Jun. 23, 2007, which claims priority from German Application Number 102006030382.2, filed Jun. 29, 2006, the disclosures of which are hereby incorporated by reference herein in their entirety.

### FIELD OF THE INVENTION

The invention relates to a solution for optical detection of the eyes.

Pathologically altered cells have altered metabolisms and gene activities, which, for example, manifest themselves in a change of the surface structure of the cells (so-called disease-correlated molecular markers). For quite some time, respective findings from molecular biological basic research have already been part of in-vitro diagnostics.

However, for the integration of in-vitro techniques and methods in in-vivo environments, a number of problems must be overcome (e.g., toxicity, targeted transport to target cell, anatomical transport barriers).

For the detection of cellular parameters relevant for the diagnosis, mainly antibody technologies and peptide chemical methods are applied, which are coupled with an imaging method.

Basically, the following elements are required:

1. Identification substance (e.g., antibody or peptide), which binds high specifically to the altered cell structures;

2. Contrast agent, which is coupled to the carrier molecule (e.g., radionucleotide or fluorescent dye);

3. Image-producing optical imaging methods for visual presentation.

With the help of molecular imaging, biological processes can be measured and characterized on a cellular and molecular level in living organisms (in vivo) [2]. Compared to standard diagnostic imaging methods, whereby anatomical features or effects of a certain disease are detected, biological processes, which underlie the disease, are detected on a cellular level. This way, diseases can be already detected at an early stage and, ideally, be treated before the appearance of the actual clinical picture.

The use of molecular imaging methods in ophthalmology is still largely unknown. For one, this is due to the fact that the molecular causes for diseases of the eye and, therefore, also the potential target molecules for the markers have only been known for a few years; second, no solution has been offered so far regarding the introduction of a molecular marker into the eye, which, on one hand, increases the dispersion or absorption of various layers or structures for the diagnosis, and on the other hand, does not worsen the functionality of the eye.

In ophthalmology it is known that with the methods of the optical coherence tomography (OCT) path lengths in the eye can be measured quite accurately. For example, with the IOLMaster from Carl Zeiss Meditec AG (www.meditec.zeiss.com) path lengths in the eye can be determined with a resolution of only a few μm. With the help of scanners, for example, Stratus-OCT and Visante OCT from Carl Zeiss Meditec AG, two- and three-dimensional images of the retina or the anterior chamber of the eye can be realized, following the same basic principle.

Through the use of infrared wavelengths (reduced dispersion of light with longer wavelengths), the OCT techniques allow for a relatively deep view into living tissue with considerable accuracy of up to 1 μm depth resolution.

Since the image contrast essentially depends on the dispersion and absorption of short-coherent light from the tissue, the sensitivity and accuracy of the measurements are greatly dependent on those optical properties of the biological tissue.

According to a review by Changhuei in [1], the sensitivity and accuracy of OCT measurements on biological tissue can be increased by using additional molecular contrast agents. In principle, there are two types of molecular contrast-based OCT's (MCOCT, in short). The first approach requires the use of appropriate, in vivo existing contrast agents, such as deoxy- and oxyhemoglobin as well as melanin. This method is only effective for a very limited number of molecules. The second approach uses additional contrast agents, which are functionalized in such a way that they bind specifically with the interesting target molecules.

US 2005/0036150 A1 describes an OCT method, which uses so-called molecular contrast agents. Thereby, molecules, which are excited energetically differently, are used in order to achieve different OCT image contrasts. However, the molecules must be optically excited when temporally coupled for the OCT diagnosis in order to produce the respective OCT contrasts. Thereto, altogether four individual methods are described in order to achieve an optical contrast increase necessary for the OCT evaluation, compared to a natural contrast due to the molecule selection.

Today, the OCT method offers the possibility of producing two- and three-dimensional images of the ocular fundus with high resolution and, therefore, diagnose changes in the retina. However, the disadvantage is that disease-relevant changes in an OCT image are only visible once the disease has broken out. Furthermore, anomalies detected in an OCT image do not necessarily have pathological causes (problem of structure and function).

However, aside from the described OCT technique, other techniques, which are based on fluorescence or bioluminescence, are also used in ophthalmology.

With fundus photography, fluorescence techniques are used, which are based on differently applied agents, for example, fluorescein (FA) or indocyanine green (ICG). This way, blood vessels in particular can be made quite visible in angiography. Also, natural pigments, such as xanthophyll (macula pigment), show a particular characteristic in the green/blue spectral region, which is used for detection.

Aside from fluorescein, used since the 60's, indocyanine green is increasingly used as dye in fluorescence angiography for the ocular fundus. While fluorescein remains the standard dye for diabetic retina changes, retinal vessel occlusions or macular edemas, ICG is increasingly used for age-related macular degeneration and other subretinal diseases, due to the limited diagnostic information from fluorescein angiography for technical reasons.

The additional information gained with ICG can be derived from the different chemical and physical properties. While fluorescein is excited with a laser with a wavelength of 480 nm, a laser with a wavelength of 800 nm is used with ICG. This light with longer wavelength penetrates the retinal pigment epithelium and also minor intraretinal and subretinal blood accumulations. Compared to fluorescein, ICG does not leave the choriocappilaris, which, in combination with a better penetration of the retinal pigment epithelium, allows for a viewing of the choroidal structures. Since ICG shows only a negligible blood concentration even after 10 cycle times, reverse effects on the images are already visible after 12 to 18 minutes.

Modern devices, such as the scanning laser opthalmoscope HRA from Heidelberg Engineering GmbH, allow for a simultaneous use of both dyes without dangerous light exposure for the patient.

A combined fluorescein and indocyanine green angiography is used particularly for the following clinical pictures:

1. Age-related macular degeneration:

For classification (dry/classic/occult) as well as improved representation of occult membranes and feeder vessels.

2. Chorioretinopathia centralis serosa:

For representation of the leaking point on the choroidal vessel, detection of previous leaking points and scars as well as membrane detection and activity control.

3. Chorioretinitis/Pigment epithelitis:

Helpful for differentiating the individual diseases through different representation in the earlier and later ICG images.

4. Macroaneurysm.

For determining the size and position of the aneurysm as well as control after coagulations.

Despite extensive studies, many phenomena of the ICG angiography are still not quite understood. Therefore, when compared to FA angiography, there is still no uniform terminology for the findings of an ICG angiography. Currently, ICG angiography can only be evaluated in combination with an FA angiography.

The described known methods for increasing contrast in ophthalmology (ICG or fluorescence angiography) are limited to contrasting blood vessels through attachment of fluorescence dyes to blood components, such as hemoglobin and albumin. Even though this allows for detection of changes in the blood vessels, e.g., neovascularization, provided they are already in an advanced stage, a detection of disease-relevant molecules and cells as well as morphological changes in tissues and membranes, as would be necessary for early detection, is not possible.

### LITERATURE

[1] Yang C., “Molecular Contrast Optical Coherence Tomography: A Review,” Photochemistry and Photobiology, 2005, 81: 215-237.
[2] Ntziachristos V., Ripoll J., Wang L V., Weissleder R., “Looking and listening to light: The evolution of whole-body photonic imaging,” Nature Biotechnology, 2005 March, 23(3): 313-20.
[3] Chen J. Saeki F., Wiley B J. Chang H., et al., “Gold Nanocages: Bioconjugation and Their Potential Us as Optical Imaging Contrast Agent,” NanoLetters 2005, Vol. 5, No. 3, 473-477.
[4] Leal E. C., Santiago A. R., Ambrosio A. F., “Old and new drug targets in diabetic retinopathy: From biochemical changes to inflammation and neurodegeneration,” Current Drug Targets—CNS & Neurological Disorders, 2005, 4(4), 421-34.
[5] Felinski E. A., Antonetti D. A., “Glucocorticoid regulation of endothelial cell tight junction gene expression: Novel treatments for diabetic retinopathy,” Current Eye Research, 2005, 30(11): 949-957.
[6] Klein M. L., Francis P. J., “Genetics of age-related macular degeneration,” Ophthalmol Clin North Am., 2003, 16(4): 567-574.
[7] Anderson D. H., Mullins R. F., Hageman G. S., Johnson L. V., “A role for local inflammation in the formation of drusen in the aging eye,” American Journal of Ophthalmology, 2002, 134(3): 411-431.
[8] Wegewitz U., Gohring I., Spranger J., “Novel approaches in the treatment of angiogenic eye disease,” Current Pharmaceutical Design, 2005, 11(18): 3211-2330.

### SUMMARY OF THE INVENTION

The invention hereto is based on the task of presenting a solution for the optical detection of changes of the eye, with which the selectivity, specificity, accuracy, and the contrast of optical measuring and diagnostic techniques for the eye is significantly increased through the use of molecular markers in order to provide a more accurate, disease-specific diagnosis already at the early stages of diseases as well as monitor the progressions of therapies.

With the solution, according to the invention, for the optical detection of changes of the eye, a molecular marker with spectral characteristics of absorption and/or dispersion in the visual and infrared spectral region or of fluorescence or bioluminescence is introduced into the eye and bound to a specific target area. The interaction of said molecular marker with the target area is detected by means of optical imaging methods, such as fundus photography, confocal laser microscopy, polarization-optical imaging methods, holographic methods, or, especially, OCT methods.

Therefore, the invention offers the advantage of an improvement of the diagnostic possibilities, particularly,
regarding the course of the disease, an early detection of defects and pathological changes;
monitoring of the success of therapeutic measures;
use of the method in medical basic research and pharmaceutical research.

The use of optical methods is strongly preferred for the diagnosis of the eye as a result of the high transparency of the optical system of the eye compared to other body parts. On the other hand, the added molecular markers, which selectively improve the optical contrast for the diagnosis, also influence the normal vision of the patient.

### BRIEF DESCRIPTION OF THE DRAWINGS

In the following, the invention is further described with the use of embodiments. They show:

FIG. 1: A schematic representation for coupling a molecular marker to a target area.

FIG. 2: A possible OCT image of a retina with molecular markers bound to target areas.

FIG. 3: A tabular overview of the applicable identification substances and contrast agents in dependency of the applied optical imaging method.

FIG. 4: A tabular overview of the targets preferably used for various diseases;

FIG. 5: An overview of currently preferred targets and the detectable eye diseases thereto;

FIG. 6: A schematic representation regarding the effect of molecular markers for diabetic retinopathy;

FIG. 7; Molecular markers for different targets for the detection of diabetic retinopathy;

FIG. 8: Molecular markers for the detection of age-related macular degeneration;

FIG. 9: Molecular markers for the detection of stem cells;

FIG. 10: Molecular markers for the detection of morbus Alzheimer syndrome; and

FIG. 11: Molecular markers for the detection of glaucoma.

### DETAILED DESCRIPTION

In the method for optical detection of changes of the eye, according to the invention, a molecular marker with spectral characteristics of absorption and/or dispersion in the visual and infrared spectral region or of fluorescence or luminescence is introduced into the eye and bound to a specific target. The interaction between the molecular marker and the target is detected by means of optical imaging methods. Since the molecular, physiologically compatible marker exhibits the characteristics of a temporally limited, selective binding to the targets in the eye with subsequent internal degradation in the body without noticeable impairment of the vision of the patient, only a slight strain to the patient and, particularly, the eye is achieved, which is adequate for diagnostic purposes.

The molecular marker, functioning as diagnostic reagent, can be injected in the patient, applied orally, or administered as eye drops. After the time period T₀, when the molecular marker has been resorbed by the body and attached itself specifically to certain targets in the target area, e.g., the retina, detection is executed with optical imaging methods. Due to the altered optical properties, the interesting molecular changes are “visible” in the image. The findings can be determined by the physician, another qualified medical employee but also through a findings software with image recognition. After a respective clearance time T_{C}, the molecular marker is either absorbed by or excreted from the body.

According to the invention, the molecular marker consists of an identification substance for high specific binding to the targets, and an optically detectable contrast agent, which is coupled to the identification substance, whereby molecules or cells, such as antibodies, peptides as well as DNA or RNA molecules, are used as identification substance. The applied identification substances can be available in the original form or in a biochemical, biotechnological or other form which was technologically altered; particularly with antibodies, the use of functional antibody fragments is feasible. The identification substances can bind specifically to the target molecules by means of hydrogen bonds, electrostatic forces, Van der Waals forces, or hydrophobic interactions, among others. The contrast agent can be bound either directly to the identification substance by means of a chemical compound, or indirectly, e.g., by means of a secondary antibody. Furthermore, binding of identification substance and contrast agent to nanoparticles, liposomes, or other biological or chemical substances as well as the insertion in such substances is possible.

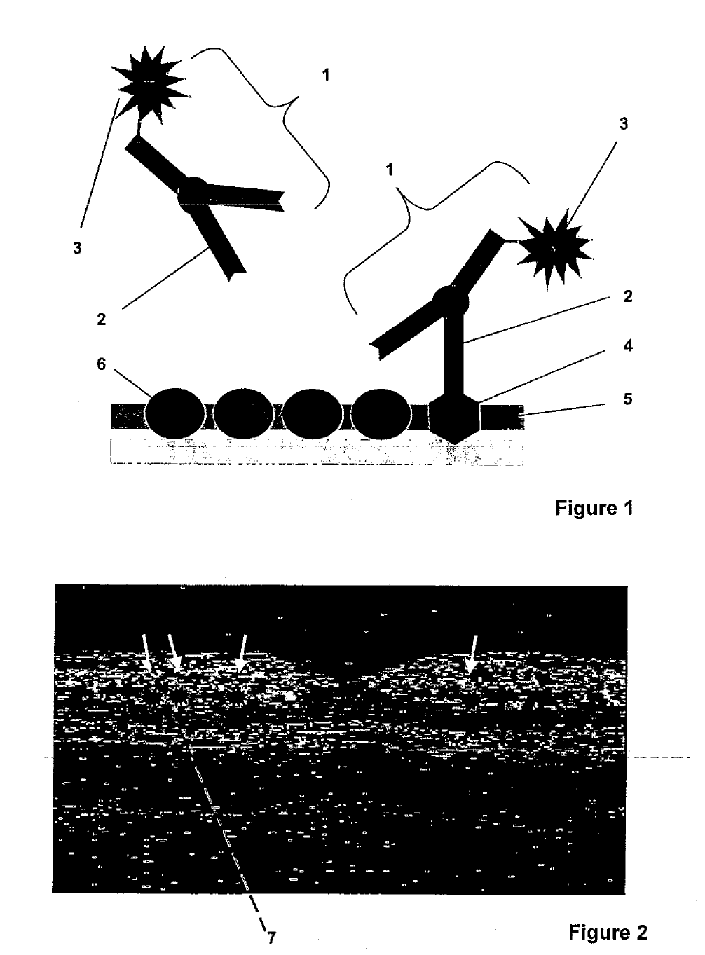

FIG. 1 shows a schematic representation for coupling a molecular marker to a target. Hereby, the molecular marker 1 consists of an identification substance 2 and contrast agent 3, coupled with the identification substance 2. The molecular marker 1 is introduced to the eye and binds with target 4. Thereby, target 4 is an altered molecule present in a membrane 5. No binding occurs with the unaltered molecules 6 inside the membrane.

The interaction between molecular marker and target is detected by means of the fundus photography, confocal laser microscopy, OCT techniques as well as other polarization or holography-based optical imaging methods. Thereto, FIG. 2 shows a possible OCT image of a retina with molecular markers bound to target areas, whereby in those areas, onto which the molecular markers are bound, distinct changes 7 in the OCT image are visible.

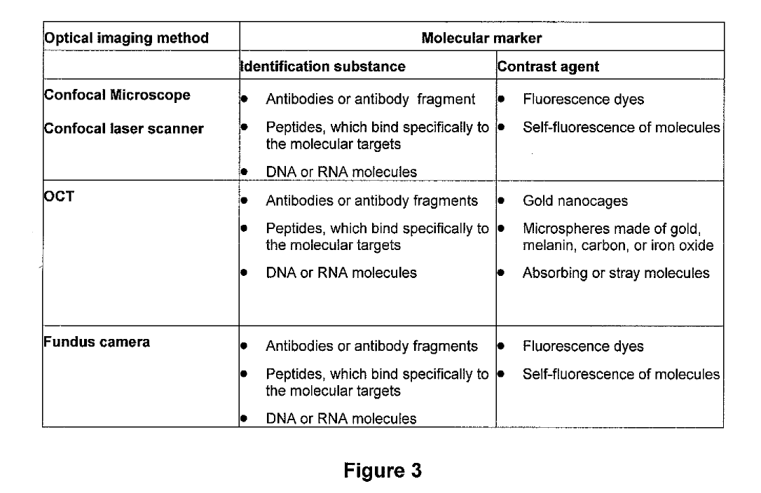

While contrast agents, which are based on fluorescence or self-fluorescence, are used for the fundus photography or confocal laser microscopy as optical imaging methods, contrast agents, which are based on light dispersion, are used for the OCT technique. Thereto, FIG. 3 shows a tabular overview of the applicable identification substances and contrast agents in dependency of the applied optical imaging method.

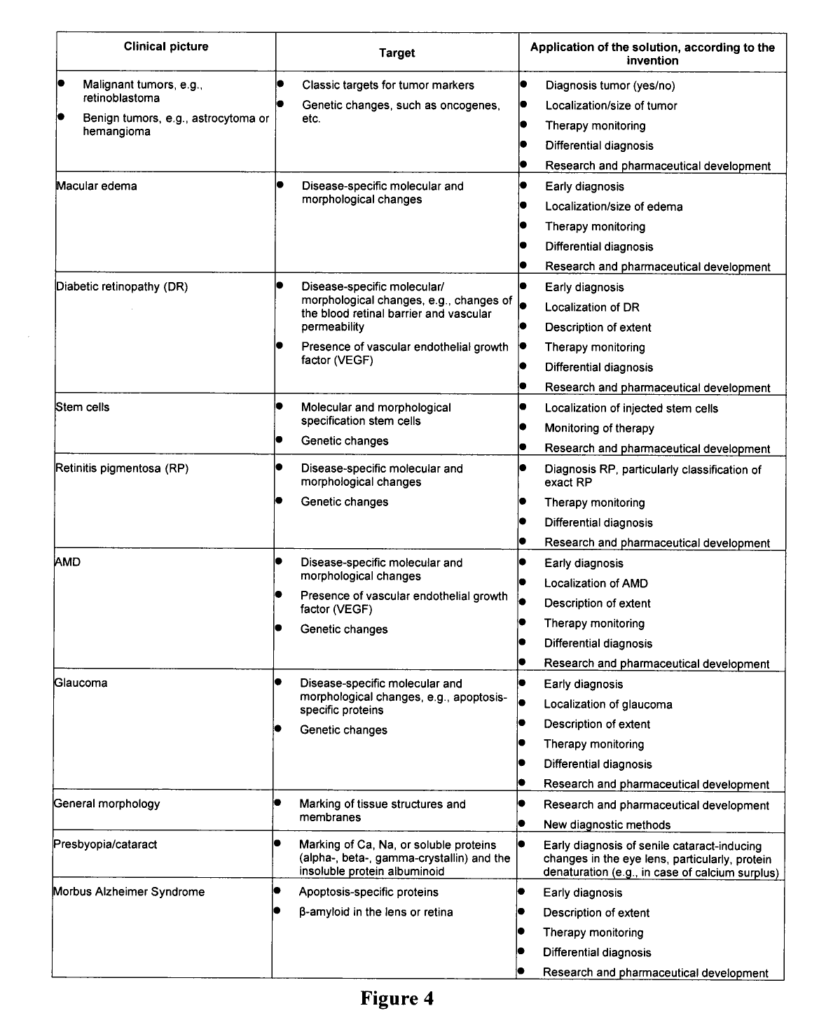

The tabular overview in FIG. 4 shows targets preferably used for various diseases, whereby the listed targets can be detected with all optical imaging methods and contrast agents listed in FIG. 3. Monoclonal or polyclonal antibodies serve as identification substance hereto. The use of peptides or DNA or RNA molecules as identification substances is also feasible. Since more and more targets and molecular causes for hereditary diseases are found within the course of medical molecular biological basic research, the tabular overview in FIG. 4 only shows the currently used and preferred targets. The list does not claim to be complete and should not be considered limiting.

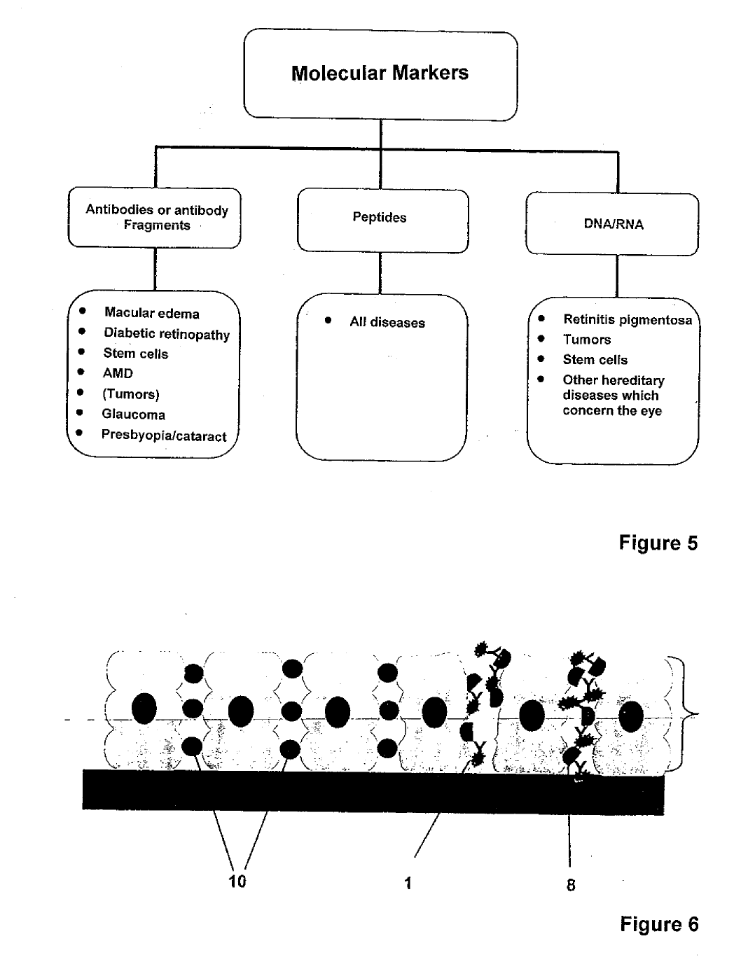

In addition, FIG. 5 shows an overview of currently used and preferred targets and eye diseases detectable with said targets.

In the following, the method, according to the invention, will be used as a more detailed example for the detection of diabetic retinopathy. According to an article by E. C. Leal and others [4], homeostasis is essential for normal retinal function. It is maintained through the blood-retina barrier (BRB), which controls the flow of water and dissolved substances to the retinal parenchyma and protects the retina from cells and antibodies from the blood.

The BRB is, among others, composed of retinal endothelial and epithelial cells, which are connected through so-called tight junctions. Those electron microscopically visible tight junctions cause the merging of the leaflets of the plasma membranes of two adjacent cells and bind those together tightly. Those tight junctions form a selective barrier for dissolved substances and allow the organism control of the transport of nutrients and degradation products.

The tight junctions consist of various transmembrane proteins, such as occluding, the junctional adhesion protein (JAM), or zonula occludens (ZO-1, ZO-2, ZO-3).

A characteristic of diabetic retinopathy is the loss of integrity and vascular permeability of the blood-retina barrier (BRB). Even in the early phases, changes of the BRB occur, which can lead to the development of macular edemas and, subsequently, to loss of vision.

According to E. A. Felinski and D. A. Antonetti in [5], diabetes thereby induces mainly the following changes:
Change of the phosphorylation of the tight junction proteins;
Spatial change in the organization of the tight junction proteins;
Decrease in the concentration of occludins.

Furthermore, in the early phases of diabetic retinopathy, the concentration of the vascular endothelial growth factor (VEGF) is greatly increased. VEGF belongs to a family of angiogenic growth factors, whereby the growth of small blood vessels (capillaries) is described as angiogenesis. An increased VEGF concentration is verifiably connected with an increased vascular permeability. Furthermore, with diabetic retinopathy, which recently has also been viewed as a chronic inflammatory disease, the cytokine levels IL-1β, IL-6, and IL-8, for example, are significantly increased, particularly in proliferative diabetic retinopathy.

Thereto, FIG. 6 shows a schematic representation regarding the effect of molecular markers in diabetic retinopathy. While the molecular marker 1 with applied antibodies penetrates through defective tight junctions 8 at the BRB 9 and recognizes disease-specific changes of the tight junctions, the molecular markers 1 are stopped at the intact tight junctions 10. Basically, it must be taken into consideration that at an intact BRB, no antibodies can penetrate. However, if the BRB is damaged, the antibodies, as shown in FIG. 6, can increasingly penetrate and be used for an increase in contrast. This effect is an example for the excellent sensitivity and specificity of the solution, according to the invention. Contrary to ICG and fluorescein angiography, the method described herein leads to a specific concentration of molecular markers at the point of the pathological change.

Other molecular targets, such as cytokines or also VEGF, detection is possible directly in the blood and, particularly, in the newly formed, pathological small blood vessels (neovascularization) without having to pass the BRB. However, VEGF can also be detected in tissue.

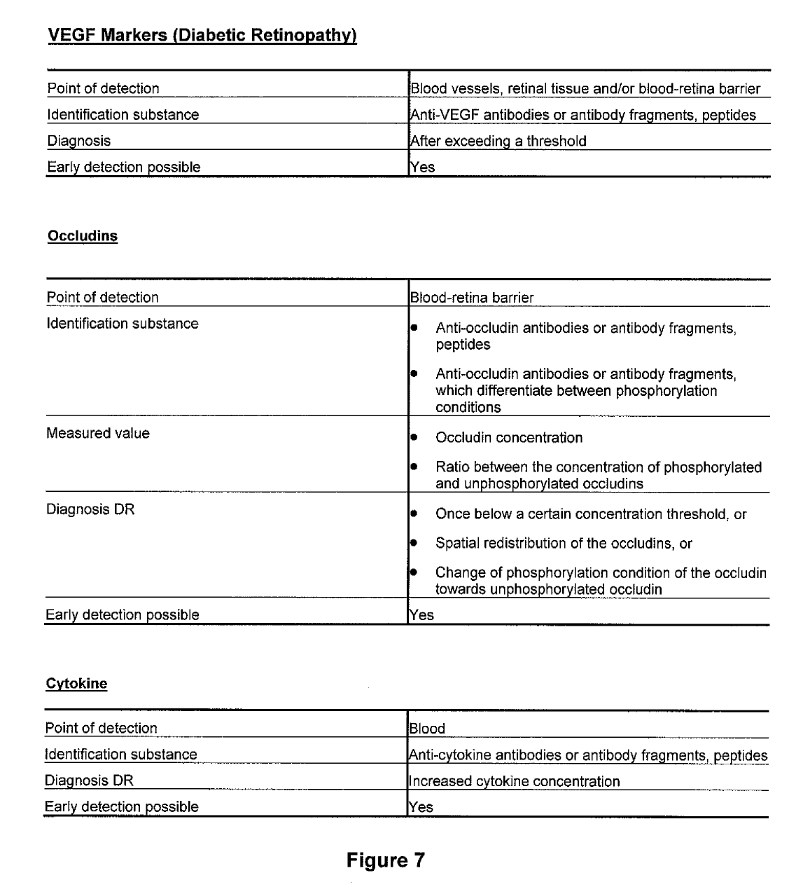

In the following, it will be explained which substances are particularly suitable as targets. As already mentioned, new targets and molecular causes for hereditary diseases are constantly discovered within the course of medical molecular biological basic research. However, currently, VEGF, occludin and the status of occludin phosphorylation as well as cytokine are particularly suited as targets. Thereto, FIG. 7 shows tabular overviews of molecular markers for different targets for the detection of diabetic retinopathy.

In the following, the method, according to the invention, will be explained, as an example, for the detection of age-related macular degeneration (AMD).

According to the article by M. L. Klein and P. J. Francis [6], AMD is one of the main causes for blindness in the western world. The pathogenesis of AMD is still not exactly known. Popular hypotheses assume that aside from an insufficient choroidal blood flow in the macula, a metabolic dysfunction of the retinal pigment epithelial or an abnormality of Bruch's membrane (membrane complex between the retinal pigment epithelial and the choroid) are causes for AMD.

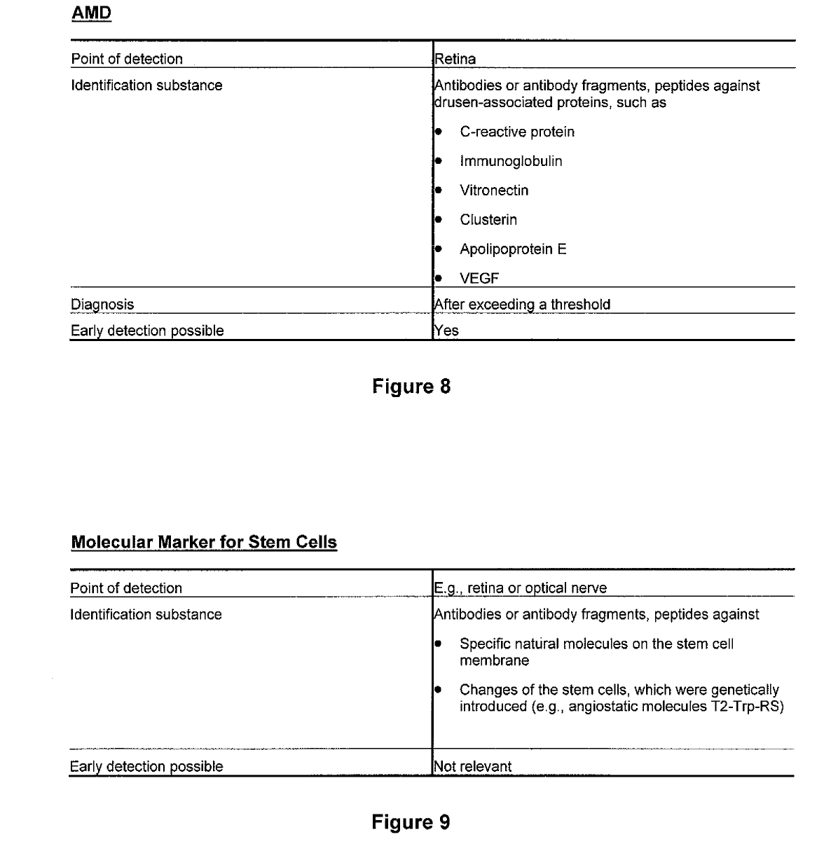

According to D. H. Anderson and others [7], the best-known morphological changes are metabolic deposits, so-called drusens. There is some evidence that inflammatory reactions play a role in drusen biogenesis, similar to Alzheimer's and atherosclerosis. There are some drusen-associated proteins, which can serve as molecular markers for AMD. FIG. 8 shows a molecular marker for the detection of age-related macular degeneration.

A particularly advantageous embodiment poses the question, to what extent a stem cell therapy can be used for curing degenerative diseases of the retina or the optical nerve.

Stem cells are body cells, which are not yet fully differentiated. In other words, they have not yet taken on a form which specializes them for the use in the organism (for example, as skin cell or liver cell), therefore, their future use is still undecided. Thereby, it is very useful for the monitoring of the therapy to observe the stem cells with the help of a detection system. This is conceivable through marking of the stem cells with specific antibodies. Thereto, FIG. 9 shows a molecular marker for the detection of stem cells.

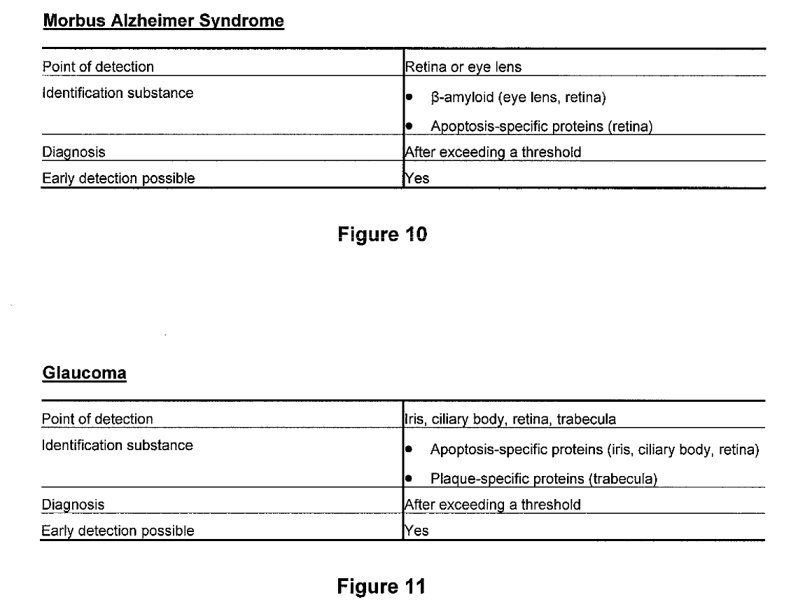

In a further advantageous embodiment, the suggested technical solution for the optical detection of the eye can be used to detect Alzheimer's disease (morbus Alzheimer syndrome) at an early stage. Alzheimer's disease, which predominantly occurs at an old age, is a disease characterized by progressive dementia of the brain, and which is associated with a progressive decrease in brain function. The disease starts with slight, apparently random forgetfulness and ends with loss of mind. FIG. 10 shows molecular markers for morbus Alzheimer syndrome as well as possible points of detection.

In a further advantageous embodiment, the suggested technical solution can also be used for detection of glaucoma. Glaucoma is one of the most frequent diseases of the optical nerve, subsequently causing characteristic losses in the visual field (scotomas), which in extreme cases lead to blindness. Glaucoma is one of the most frequent causes for blindness in industrial countries as well as developing countries. Based on the points of detection, FIG. 11 shows molecular markers for the detection of glaucoma.

The device for the optical detection of changes of the eye, according to the invention, consists of an optical imaging unit for the detection of the interaction of a molecular marker, introduced to the eye and bound to a specific target, and an evaluation unit, whereby the molecular marker exhibits a spectral characteristic of absorption and/or dispersion in the visual and infrared spectral region or of fluorescence or bioluminescence.

Since the molecular, physiologically compatible marker also exhibits the characteristics of a temporally limited, selective binding to the targets in the eye with subsequent internal degradation in the body without noticeable impairment of the vision of the patient, only a slight strain to the patient and, particularly, the eye is achieved, which is adequate for diagnostic purposes.

As already mentioned, the molecular marker, functioning as diagnostic reagent, can be injected in the patient, applied orally, or administered as eye drops. After the time period T₀, when the molecular marker has been resorbed by the body and attached itself specifically to certain targets in the target area, e.g., the retina, detection is executed with optical imaging methods. Due to the altered optical properties, the interesting molecular changes are “visible” in the image. The findings can be determined by the physician, another qualified medical employee but also through a findings software with image recognition. After a respective clearance time T_{C}, the molecular marker is either absorbed by or excreted from the body.

According to the invention, the molecular marker consists of an identification substance for high specific binding to the targets, and an optically detectable contrast agent, which is coupled to the identification substance, whereby molecules or cells, such as antibodies, peptides as well as DNA or RNA molecules, are used as identification substance. The applied identification substances can be available in the original form or in a biochemical, biotechnological or other form which was technologically altered; particularly with antibodies, the use of functional antibody fragments is feasible. The identification substances can bind specifically to the target molecules by means of hydrogen bonds, electrostatic forces, Van der Waals forces, or hydrophobic interactions, among others. The contrast agent can be bound either directly to the identification substance by means of a chemical compound, or indirectly, e.g., by means of a secondary antibody. Furthermore, binding of identification substance and contrast agent to nanoparticles, liposomes, or other biological or chemical substances as well as the insertion in such substances is possible.

The interaction between molecular marker and target is detected by means of fundus cameras, confocal laser microscopes, OCT devices as well as other polarization or holography-based optical imaging devices. While contrast agents, which are based on fluorescence or self-fluorescence, are used for optical imaging by means of fundus cameras or confocal laser microscopes, contrast agents, which are based on light dispersion or absorption, are used for the OCT devices.

In an embodiment, the optical imaging unit is a device based on optical coherence tomography (OCT). Hereby, the molecular marker exhibits increased absorption and/or dispersion in the infrared spectral region and a lowest possible absorption and/or dispersion in the visual spectral region. Hereby, in particular, the molecular marker of the operating wavelength of the OCT device should exhibit increased absorption and/or dispersion. Due to the low absorption and/or dispersion in the visual spectral region, the lowest possible impairment of the vision of the patient is enabled.

In another embodiment, the optical imaging unit is a confocal laser microscope or confocal laser scanner. Particularly, with the applied laser wavelength, the molecular marker exhibits thereby increased absorption and/or dispersion or fluorescence or bioluminescence in the visual or infrared spectral region.

The described standard use of a confocal scanner for the spatially resolved detection of the molecular markers can, according to the invention, possess an additional temporally resolved detection. This allows for spatial and temporal resolution, e.g., evaluation of the fluorescence decay time of the dye molecule bound to the marker molecule.

A fluorescence lifetime can, spatially resolved, be assigned to individual detection points, and therefore, also produce images. Since those decay times depend on the condition of the binding, it becomes apparent whether or not binding conditions or specific bindings have taken place in the examined spatial areas.

Thereto, methods of confocal microscopy and optical coherence tomography cannot only be used as two- and three-dimensional imaging methods. With a linear scan (e.g., A-scan) with the introduced molecular marker in the target area, both methods can also provide a specific signal, which characterizes the binding condition and, therefore, allows for a diagnosis. With this simplified diagnosis, e.g., in the lens, not only the anatomical boundary layers of the lens are visible as peak in the scan, but also the marker-specific peaks, which characterize the specific binding and presence.

In a further embodiment, the optical imaging unit is a fundus camera, and with the applied excitation wavelength range, the molecular marker exhibits thereby either increased fluorescence and/or bioluminescence in the visual or infrared spectral region. The detection of the interaction of the molecular marker, which was introduced into the eye and bound to a specific target, takes place in a respective spectral region with longer waves. However, it is also possible that with the applied excitation wavelength range, the molecular marker exhibits increased absorption and/or dispersion in the visual or infrared spectral region. Therefore, the detection of said interaction takes place in the visual or infrared spectral region.

At a given intensity threshold of the camera system, including camera chip, with a threshold factor “IS” and the known reflectivity of the retina of approximately 10⁻⁴, the natural contrast of retina images, for example, from a fundus camera is caused because the illumination intensity is >10⁻⁴×IS. The marker-specific fluorescence signals for a respective fluorescence image must be particularly distinguishable from the autofluorescence signal with the respective combination of excitation wavelength and detection wavelength, Since the fluorescence dyes used in the marker are adjusted to the respectively used excitation and detection wavelengths of the optical diagnostic system, a clear useful signal is expected when compared to the autofluorescence background. Thereby, with a comparatively low radiation level, a greatly increased useful signal is achieved with the marker-bound molecular diagnosis, according to the invention, than with a molecular imaging, which, e.g., is based on autofluorescence, fluorescence lifetime, or Raman molecular diagnosis method without additional markers. Despite the disadvantage of having to introduce a marker into the eye, this characteristic is of particular importance for the eye, since radiation threshold values have to be strictly observed in order to protect the retina.

The solution, according to the invention hereto, uses alternatively absorption, dispersion, or fluorescence as optical contrast enhancement, which are selectable through the contrast agents bound to the molecular markers.

1-18. (canceled)

19. A method for the optical detection of the eye of a patient, comprising:
introducing a physiologically compatible molecular marker that binds to a specific target area into the eye, the molecular marker having spectral characteristics of absorption and/or dispersion in the visual and infrared spectral region or of fluorescence or bioluminescence; and
detecting the interaction of said molecular marker with said target by optical imaging methods.

20. The method according to claim 19, further comprising selecting the, physiologically compatible molecular marker such that it exhibits temporally limited, selective binding to the targets in the eye with subsequent internal degradation in the body without noticeable impairment of the vision of the patient.

21. The method according to claim 19, further comprising selecting the molecular marker to include an identification substance for high specific binding to the targets, and an optically detectable contrast agent, which is coupled to the identification substance.

22. The method according to claim 21, further comprising selecting the molecular marker to include molecular or cellular identification substances selected from a group consisting of antibodies, peptides, DNA molecules and RNA molecules.

23. The method according to claim 19, further comprising detecting interaction of the molecular marker with the target by a technique selected from a group consisting of fundus photography, confocal laser microscopy, OCT technique, polarization-based optical imaging methods and holography-based optical imaging methods.

24. The method according to claim 19, further comprising dynamically determining additional wavefront data of the optical system of the individual eye to compensate for aberrations of the eye to enable high resolution detection.

25. The method according to claim 23, further comprising using fluorescent contrast agents based on fluorescence or self fluorescence, along with the fundus photography or the confocal laser microscopy as optical imaging method.

26. The method according to claim 19, further comprising using contrast agents based on light dispersion and using OCT technique as the optical imaging method.

27. The method according to claim 19, in which the specific target areas in the eye comprise molecules or cells which differ from healthy molecules or cells due to pathological changes.

28. The method according to claim 19, further comprising using antibodies which bind to cytokine, occludin or VEGF and act as molecular markers for the detection of diabetic retinopathy.

29. The method according to claim 19, further comprising using antibodies which bind to drusen-associated proteins for detection of age-related macular degeneration.

30. The method according to claim 29, further comprising selecting the antibodies to bind to the drusen related proteins selected from a group consisting of C-reactive proteins, immunoglobulin, vitronectin, clustering and apolipoprotein E.

31. The method according to claim 19, further comprising using antibody fragments or peptides which bind to apoptotic proteins or β-amyloid as molecular markers for detection of morbus Alzheimer syndrome.

32. A device for the optical detection of the eye, comprising:
an optical imaging unit that detects interaction of a molecular marker introduced into the eye and bound with a specific target; and
an evaluation unit;
wherein the molecular marker exhibits a spectral characteristic of absorption and/or dispersion in the visual and/or infrared spectral region or of fluorescence or bioluminescence.

33. The device according to claim 32, wherein the optical imaging unit comprises a device based on optical coherence tomography (OCT), and the molecular marker exhibits increased absorption and/or dispersion in the infrared spectral region and a reduced absorption and/or dispersion in the visual spectral region relative to the absorption and/or dispersion in the infrared spectral region.

34. The device according to claim 32, wherein the optical imaging unit comprises a confocal laser microscope and the molecular marker exhibits increased absorption and/or dispersion or fluorescence or bioluminescence in the visual or infrared spectral region in response to laser energy applied by the confocal laser microscope.

35. The device according to claim 32, wherein the optical imaging unit comprises a fundus camera and the molecular marker exhibits increased fluorescence and/or bioluminescence in the visual or infrared spectral region in response to excitation light energy in an excitation wavelength range applied by the fundus camera.

36. The device according to claim 32, wherein the optical imaging unit comprises a fundus camera and the molecular marker exhibits increased absorption and/or dispersion in the visual or infrared spectral region in response to excitation light energy in an excitation wavelength range applied by the fundus camera.

37. The device according to claim 32, wherein the optical imaging unit contains an adaptive optical system or a phase plate system, with which aberration of the eye is compensated for to enable highest-resolution detection.

38. The device according to claim 37, wherein the adaptive optical system or a phase plate system compensates for the aberration of the eye based on dynamically determined wavefront data of the optical system of an individual eye
